(19) United States
(12) Patent Application Publication
(10) Pub. No.: US 2018/0000869 A1
(43) Pub. Date: Jan. 4, 2018

(54) AMNIOTIC FLUID-DERIVED PREPARATIONS

(71) Applicant: Applied Biologics, LLC, Scottsdale, AZ (US)

(72) Inventor: Edward Britt, Scottsdale, AZ (US)

(73) Assignees: Applied Biologics, LLC, Scottsdale, AZ (US); Applied Biologics, LLC, Scottsdale, AZ (US)

(21) Appl. No.: 15/637,869

(22) Filed: Jun. 29, 2017

Related U.S. Application Data
(60) Provisional application No. 62/356,323, filed on Jun. 29, 2016.

(51) Int. Cl. A61K 35/50; A61K 47/10; A61K 38/43; A61K 35/36; A61K 35/28; A61K 47/20; A01N 1/02

(52) U.S. Cl. CPC A61K 35/50; A61K 47/20; A61K 47/10; A01N 1/0221; A61K 35/36; A61K 35/28; A61K 38/43

(57) ABSTRACT

The invention relates to an amniotic fluid-derived preparation. The amniotic fluid-derived preparation leverages cellular and non-cellular constituent components of amniotic fluid for use across a broad range of therapeutic applications, including use by physicians and other healthcare providers in the surgical and minimally invasive medical therapy of a wide range of injuries and disease processes. The amniotic fluid-derived preparation concentrates available quantities of non-cellular bioactive proteins and cellular elements to enhance therapeutic efficacy in multiple clinical settings. The amniotic fluid-derived preparation may be intraoperatively transplanted at the recipient site using a needleless syringe, by non-operative percutaneous injection through a hypodermic needle, or by direct application.

### CROSS-REFERENCE TO RELATED APPLICATIONS

This application claims the benefit of and priority to U.S. Provisional Patent Application No. 62/356,323, filed Jun. 29, 2016, the entire contents of which are incorporated herein by reference.

### BACKGROUND OF THE INVENTION

### Technical Field

This invention relates to preparations derived from amniotic fluid, and more specifically, to amniotic fluid-derived preparations used in clinical and research applications.

### State of the Art

Amniotic fluid, specifically human amniotic fluid, has been identified as a rich source of therapeutic biomolecules. Amniotic fluid's suspended protein fraction and cellular components contain a complex biologic soup of growth factors, inflammatory regulators, immuno-modulators, and other active biomolecules. Amniotic fluid is rich in pluripotent cellular elements, including amniotic fluid stem cells which also contain high intracellular concentrations of regulatory proteins and other biologically active substance that are secreted into the extracellular milieu where therapeutically relevant bioactivity is mediated through paracrine “action-at-a-distance” signaling mechanisms.

Amniotic fluid derivatives, with or without a cellular component, have tremendous potential for use in a range of clinical and medical research applications. An amniotic fluid-derived product, however, must be processed and concentrated in a manner which preserves protein bioactivity and cellular viability, quantified with respect to specific protein and cellular components, and then packaged for convenient and practical use by a clinician or research scientist. Without a standardized amniotic fluid-derived product, clinical use—including clinical trials in human subjects—may lead to unpredictable results or be unsafe. Furthermore, it is problematic for researchers to obtain reproducible results in research applications without such a standardized product.

Accordingly, what is needed is a reconstituted, standardized amniotic fluid-derived product which is commercially available, of consistent quality, and safe for clinical and investigational use.

Citation of documents herein is not an admission by the applicant that any is pertinent prior art. Stated dates or representation of the contents of any document is based on the information available to the applicant and does not constitute any admission of the correctness of the dates or contents of any document.

### SUMMARY OF THE INVENTION

Disclosed is an amniotic fluid preparation comprising a first protein fraction isolated from a donor amniotic fluid; a cellular component isolated from a donor amniotic fluid; and a fluid, wherein the fluid dilutes the protein fraction and the cellular component.

The donor amniotic fluid may be amniotic fluid obtained or derived from a human or another species. For example, in some embodiments, the donor amniotic fluid is a human amniotic fluid. In some embodiments, the donor amniotic fluid is amniotic fluid from a mammal, for example, a primate. In some embodiments, the donor amniotic fluid is a non-human amniotic fluid, for example a non-human mammalian amniotic fluid. In some embodiments, the amniotic fluid preparation further comprises a cryopreservative. In some embodiments, the cryopreservative comprises dimethylsulfoxide and/or glycerol.

Amniotic fluid-derived preparations of the invention may include the total collection of proteins expressed in a donor amniotic fluid at the time of fluid collection (i.e., the donor amniotic fluid proteome). In some embodiments, the first protein fraction comprises an amniotic fluid proteome. In some embodiments, the first protein fraction comprises a secondary source protein wherein the amniotic fluid proteome does not comprise the secondary source protein. In some embodiments, the first protein fraction comprises one or more concentrated regulatory proteins taken from the group of regulatory proteins consisting of a growth factor, a signaling ligand, a receptor molecule, a cytokine, a transcriptional regulator, and an immune regulator. In some embodiments, the first protein fraction comprises one or more concentrated enzymes. In some embodiments, the first protein fraction comprises one or more concentrated binding proteins. In some embodiments, the first protein fraction comprises one or more concentrated carrier proteins.

In some embodiments, a first protein fraction isolated from a donor amniotic fluid is acellular (i.e., the first protein fraction is entirely free of cells). In some embodiments, the first protein fraction isolated from a donor amniotic fluid is “cell-depleted,” i.e., nearly entirely free of cells, for example, the first protein fraction contains fewer than 10,000 cells/ml, fewer than 1,000 cells/ml, fewer than 100 cells/ml, or fewer than 10 cells/ml.

In some embodiments, the cellular component comprises an epithelial stem cell. In some embodiments, the cellular component comprises a mesenchymal stem cell. In some embodiments, the cellular component comprises a progenitor cell. In some embodiments, the cellular component comprises an epithelial cell. In some embodiments, the cellular component is substantially depleted of epithelial cells. In some embodiments, the cellular component is substantially depleted of mesenchymal cells.

Disclosed is an amniotic fluid derivative comprising a concentrated cellular component; a supernatant; and a fluid, wherein the fluid dilutes the concentrated cellular component and the supernatant.

In some embodiments, the amniotic fluid derivative further comprises a concentrated exosome component. In some embodiments, the supernatant is substantially free of exosomes. In some embodiments, the concentrated cellular component comprises a non-amniotic fluid derived cell. In some embodiments, the supernatant is substantially depleted of albumin. For example, in some embodiments, the supernatant is substantially depleted of the soluble, monomeric human protein, United States National Center for Biotechnology Information (“NCBI”) accession number CAA00606.1 (SEQ ID NO:1). In some embodiments, the supernatant is substantially depleted of immunoglobulin, for example, all immunoglobulin. In some embodiments, the supernatant can be substantially depleted of one or more immunoglobulins. In embodiments described herein, the supernatant can be completely depleted of one or more immunoglobulins.

In some embodiments, the supernatant is acellular (i.e., the supernatant is entirely free of cells). In some embodiments, the supernatant is “cell-depleted,” i.e., nearly entirely free of cells, for example, the supernatant contains fewer than 10,000 cells/ml, fewer than 1,000 cells/ml, fewer than 100 cells/ml, or fewer than 10 cells/ml.

In some embodiments, the invention includes one or more sets of amniotic-fluid derived preparations, including one or more sets of amniotic-fluid derived preparations that encompass any of the aforementioned characteristics of individual amniotic fluid-derived preparations described herein. In some embodiments, sets of amniotic-fluid derived preparations include amniotic fluid-derived preparations where each amniotic fluid-derived preparation is the same or nearly the same as every other amniotic fluid-derived preparation in the set. For example, in some embodiments, the invention includes a set of amniotic fluid-derived preparations, wherein each amniotic fluid-derived preparation includes: a first protein fraction isolated from a donor amniotic fluid; a cellular component isolated from the donor amniotic fluid; and a fluid. In such embodiments, the fluid dilutes the first protein fraction and the cellular component, and the dilution of the first protein fraction and the cellular component in each amniotic fluid-derived preparation in the set is the same or about the same as the dilution of the first protein fraction and the cellular component in every other amniotic fluid-derived preparation in the set.

In some embodiments, the invention includes a set of amniotic fluid-derived preparations, wherein each amniotic fluid-derived preparation includes: a concentrated cellular component; a supernatant; and a fluid. In such embodiments, the fluid dilutes the concentrated cellular component and the supernatant, and the dilution of the concentrated cellular component and the supernatant in each amniotic fluid-derived preparation in the set is the same or about the same as the dilution of the concentrated cellular component and the supernatant in every other amniotic fluid-derived preparation in the set.

In various embodiments, the invention may comprise a set of amniotic fluid-derived preparations that includes a minimum number of amniotic fluid-derived preparations. For instance, a set of amniotic fluid-derived preparations of the invention may include at least 1, at least 5, at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, or at least 1,000 amniotic fluid-derived preparations.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a schematic representation of an amniotic fluid-derived preparation, including its individual components;

FIG. 2 is a schematic representation of a donor amniotic fluid and its constituent supernatant and cellular components;

FIG. 3 is a schematic representation of the inter-relationship between a donor amniotic fluid, a secondary source protein, a first protein fraction, and a second protein fraction;

FIG. 4 is a schematic representation of a cellular component comprising a first cell type and a second cell type.

### DETAILED DESCRIPTION OF THE INVENTION

A detailed description of the hereinafter described embodiments of the disclosed composition are presented herein by way of exemplification and without limitation with reference to the Figures. Although certain embodiments are shown and described in detail, it should be understood that various changes and modifications may be made without departing from the scope of the claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise.

It is to be understood that some of the terms used herein to disclose the elements and various embodiments of the present invention may have broad meaning according to at least the definitions provided herein below. “Amniotic fluid” (abbreviated herein as “AF”) means fluid originating in the amniotic sac of a pregnant female and comprising suspended cellular and non-cellular elements, including all defined and undefined components, molecules, and compounds.

“Preparation” means a substance specially made up from component substances. In an example, an amniotic fluid-derived preparation is a substance specially made up from at least one or more of a group of components consisting of a cellular component, a supernatant, and a fluid. This example is not meant to be limiting.

“Cellular component” means intact cells originating in AF. Generally, the cellular component is suspended within the fluid component of AF. The cellular component includes any cell type, whether defined and known or undefined and unknown, which may be present in AF. “Cell concentration” means the number of cells present per unit volume of a fluid, such as the number of cells in a milliliter of fluid, for example. “Viable cell concentration” means a cell concentration wherein the counted cells are viable cells wherein viability is determined by a standard dye exclusion assay. A non-limiting example of a dye exclusion assay is a Trypan blue assay; other dye exclusion assays and other methods of determining cell viability may be available. “Stem cells” means undifferentiated cells which may give rise to additional generations of stem cells or which may differentiate into progenitor cells. When used in this application, “stem cell” means a stem cell originating in the cellular component of AF, however, stem cells may otherwise originate in fetal membranes, other fetal-derived tissues, or non-fetal tissues. “Epithelial stem cell” means a stem cell originating from the embryonic epithelium, including the ectoderm and the endoderm embryonic layers. “Mesenchymal stem cell” means a stem cell capable of lineage differentiation into mesenchymal lineages; for example, osteogenic, chrondrogenic, and adipogenic lineages, and originating from the embryonic mesenchyme, including stromal and vascular tissue of the umbilical cord. Wherein “stem cell” is used as referring to a stem cell not originating in the cellular component of AF, the specification will explicitly note a non-AF origin of the stem cell. “Progenitor cell” means a cell which is committed to differentiating 1) along a specific germ cell line, i.e. ectoderm, mesoderm, or endoderm; or 2) a cell committed to differentiating into a specific cell or tissue, i.e. chondrocyte or integrated cortical columnar unit.

“Relative centrifugal force” means the radial force generated by a spinning centrifuge rotor expressed relative to the earth's gravitational force. For example, a relative centrifugal force of 100 g means a radial force one hundred (100) times the force of gravity. “Supernatant” means the liquid layer layered over insoluble material after centrifugation which may be removed, such as by pipetting or decanting. The meaning of “supernatant” additionally includes any fluid layered over a solid residue following crystallization, precipitation, or other process causing the solid residue to become distinct from the covering fluid. Supernatant includes water or other liquid and all constituent materials, including compounds in solution or suspension and intact cells, cellular elements, organelles, membrane fragments, and the like remaining in suspension following centrifugation, precipitation, and the like.

“Protein fraction” means at least one protein included in AF. Protein fraction is a portion of an AF containing a protein, for example an AF supernatant. A protein fraction may comprise one protein or the entire AF proteome. A protein fraction may comprise an entire AF supernatant or any portion of an AF supernatant comprising at least one protein arising from AF. A protein fraction may comprise an additional non-AF protein from a secondary source separate from a donor AF, including an AF protein from a second donor AF, a non-AF protein, or an AF or other protein produced outside of AF by other means such as by a genetically engineered bacterium, mammalian cell, yeast baculovirus, extracellular in vitro protein synthesis, and the like.

“K_{d}” means a dissociation constant, such as the dissociation constant of an enzyme, an antibody, and the like. “Buffer solution” means an aqueous solution comprising a weak acid and its conjugate base used to stabilize the pH by resisting changes in pH when acid or base is added. A buffer solution is used to stabilize the pH of the solution within a narrow range around a specific value. “Buffer solution” is used generically herein to mean buffer solution appropriate for a given application and not one specific buffer solution. Examples of suitable buffer solutions include a phosphate buffer solution (“PBS”) and buffer solutions commonly used in biologic applications.

“Donor” means a pregnant female, including a peripartum female delivering an infant, from whom amniotic fluid is obtained. “Fetal placental membranes” is used synonymously with “fetal membranes” and means any or all of the amnion, chorion, and Wharton's jelly.

“Lyophilization” means drying by removal of water through sublimation of water ice directly to water vapor without passing through a liquid phase. “Concentrated” means a relative concentration of a cell, a protein, a non-cellular non-protein substance or other material per unit volume that is greater than the original concentration of that substance in the donor AF. “Substantially depleted” means a concentration of a cell, a protein, a non-cellular non-protein substance, or other material per unit volume of a preparation or fluid wherein the concentration is less than the concentration of that material in the donor AF from which the material is derived. For example a substantially depleted cell, protein, non-cellular non-protein substance, or other material may be about 10%, about 20%, about 30%, about 40%, about 50%, less than about 10%, about 0% to about 10%, about 10% to about 20%, about 20% to about 30%, about 30% to about 40%, about 40% to about 50%, about 0% to about 20%, or about 10% to about 30% of the concentration of that material in the donor AF from which the material is derived.

“Immunoglobulin” means any one or more specific proteins belonging to the family of proteins which may be produced by white blood cells and act as antibodies. “Albumin” means a soluble, monomeric human protein, United States National Center for Biotechnology Information (“NCBI”) accession number CAA00606.1 (SEQ ID NO:1). “Lysate” means the intracellular products released by the disruption of a cell membrane by any means, such as mechanical, chemical, or other means. “Thickening agent” means a water soluble polymer that increases the viscosity of a solution or suspension. “Hydrogel” means a colloidal gel wherein the constituent colloidal particles are dispersed in water. Unless otherwise stated, “hydrogel” means a colloidal gel with an aqueous dispersion medium. A hydrogel is an example of a thickening agent.

The disclosed invention relates to amniotic fluid-derived preparations. Specifically, embodiments of the invention comprise preparations formed from cellular and non-cellular derivatives of amniotic fluid. The disclosed embodiments of amniotic fluid-derived preparations may be used in tissue regenerative therapy, other medical therapies, and research into the treatment of multiple surgical and non-surgical degenerative conditions.

AF and its constituent components occupy a unique position in the field of regenerative medicine. The fluid, which derives from both maternal plasma and the developing embryo and fetus, comprises water, electrolytes, proteins and other classes of biologically active molecules, and cells. The cellular component includes epithelial and mesenchymal stem cells of both fetal and maternal origin.

AF may be separated into a cellular component and a supernatant. This separation is commonly accomplished by centrifugation, although other suitable means are available, such as ultrafiltration, precipitation, and the like. The cellular component includes different families of stem cells, of both embryonic and extra-embryonic (maternal) origin. AF stem cells include both epithelial and mesenchymal stem cells. Mesenchymal stem cells from AF may include cells that express any combination of CD44, CD29, CD49e, CD58, CD90 (Thy-1), CD105 (endoglin), CD73, CD166, HLA-ABC (MHC Class I), Oct-3/4, Nanog, Sox-2, stage-specific embryonic antigen 4 (SSEA-4), and Rex-1, and which do not express appreciable levels of CD34, CD45, CD31, CD40, CD14, HLA-DR (MHC Class II), latexin (LXN), growth differentiation factor 6 (GDF6), Ig mu heavy chain disease protein (MUCB), alpha crystallin B chain (CRYAB), glycogen synthase kinase 3 beta (GSK3β), and ATP dependent RNA helicase DDX 19A (DD19A). Epithelial stem cells from AF may include cells that express any combination of CD10, CD13, CD29, CD44, CD49e, CD73, CD90, CD105, CD117, CD166, Stro-1, HLA-ABC, HLA-DQ^{low}, SSEA-1, SSEA-3, SSEA-4, Nanog, sex determining region Y-box2 (Sox2), Tra1-60, Tra1-80, fibroblast growth factor 4 (FGF4), Rex-1, cryptic protein (CFC-1), and prominin 1 (PROM-1), and which do not express appreciable levels of CD14, CD34, CD45, CD49d, and HLA-DR. These stem cells are often capable of engraftment and differentiation within host tissue of another individual. AF stem cells are also capable of paracrine secretion of regenerative growth factors and other bioactive substances. Additionally, AF stem cells neither express human leukocyte Class I antigens (“HLA-I”) nor can they differentiate into hematopoietic cells. Consequently, transplanted amniocytes do not provoke an immune response in the recipient and cannot differentiate into host-sensitized T-lymphocytes capable of mounting a graft-versus-host reaction. This lack of immunogenicity makes donor AF stem cells a unique and versatile allograft.

The supernatant contains a large variety and concentration of proteins and other large and small biomolecules. In addition to albumin and immunoglobulin, multiple families of regulatory proteins are present which likely affect fetal growth, development, and interaction with the maternal physiologic environment. Growth factors secreted by the mother and fetus are the principal non-cellular active biological compounds native to amniotic fluid. Systematic evaluation of the human amniotic fluid proteome has identified numerous proteins within gene ontology (“GO”) categories relevant to tissue healing, regenerative bioactivity, and biologic augmentation. GO categories are functional identifiers of gene and protein networks that indicate the functional significance of proteins and genes naturally present in amniotic fluid. Key GO categories that have so far been identified include: 1) cellular movement; 2) development and function; 3) cellular growth and proliferation; 4) cell-to-cell signaling and interaction; 5) tissue differentiation; and 6) organism development. These GO-classifiers identify the presence of specific categories of growth factors and growth factor networks directly associated with regenerative bioactivity (Cho, et al., (2012) “Proteomic analysis of human amniotic fluid,” Mol Cell Proteomics 6:1406-15).

AF for amniotic fluid-derived preparations is potentially available in substantial quantities from a pool of donors. There are almost 4 million births per year in the United States, constituting a pool of potential AF donors. From this pool, AF is made available from a suitably screened subpopulation. Potential donors undergo a pre-donation screening process to minimize the risk of transmission of maternal or fetal infectious agents by way of donated AF to an eventual recipient of an amniotic fluid-derived preparation. This screening procedure includes subjective and objective components. The subjective component may include screening by administration of a donor questionnaire to identify high-risk social behaviors for infectious disease. Some paid donors are motivated to hide a past social history of high-risk behavior for transmission of sexually transmitted infections, including hepatitis B virus (“HBV”), hepatitis C virus (“HCV”), and human immunodeficiency virus (“HIV”). Accordingly, only volunteer donors are used. The objective component comprises (pre-delivery) laboratory screening including a metabolic panel including liver function studies and assessment of serology for evidence of past or present HBV, HCV, or HIV infection, in some embodiments.

AF from acceptable donors may be excluded by perinatal observations and events. Clinical or laboratory evidence of active maternal or fetal infection around the time of delivery, the most severe example exemplified by chorioamnionitis, precludes the use of AF. Meconium staining of the AF and/or the fetal membranes, although usually not indicative of infection, also eliminates the individual from the donor pool. Finally, and most commonly, contamination of the placental membranes with a large quantity of maternal blood, feces, or other perinatal sources of gross bacterial or tissue contamination precludes use of the AF.

Unlike fetal placental membranes, it is generally not practical to obtain AF from a donor during a vaginal delivery because, in the majority of vaginal deliveries, the placental membranes spontaneously rupture and the AF is lost. Controlled, therapeutic rupture of membranes, however, is an exception and is discussed herein below. The use of AF from donors undergoing a Cesarean-section delivery essentially eliminates gross bacterial contamination of the donor AF. Of the approximately 4 million births annually in the U.S. mentioned earlier, approximately 33%—1.32 million overall—are by Cesarean delivery which reduces the potential donor pool for AF by nearly seventy percent. AF, therefore, is potentially available to develop derived preparations from a total of between 0.95 and 1.32 million births annually in the U.S.

As noted herein above, AF may be collected from suitable volunteer donors and processed for storage prior to deriving preparations for use in a variety of surgical procedures and non-surgical clinical, and research applications. Some examples of non-surgical clinical applications include use of amniotic fluid-derived preparations in dressings and wound treatments as an adjunct to healing, particularly in the treatment of chronically ischemic or infected wounds; as a component in the creation of artificial skin, and to augment healing of tendon and ligamentous injuries. Therefore, in some embodiments, amniotic-fluid derived preparations of the invention can be used in methods of dressing and treating wounds, in methods of creating artificial skin, or in methods of augmenting healing of tendon and ligamentous injuries. Surgical uses of amniotic fluid-derived preparations include introduction as an adjunct to healing of surgically repaired bone, tendon, other soft tissue, and open wounds; a means to militate the formation of scar tissue and adhesions, and other beneficial applications in surgery and non-surgical minimally invasive medical therapies. Therefore, in some embodiments, amniotic fluid-derived preparations may be used in methods of healing surgically repaired bone, tendon, other soft tissue, and/or open wounds; in methods of militating the formation of scar tissue and adhesions; and in methods of performing surgical and non-surgical minimally invasive medical therapies. In some embodiments, amniotic fluid-derived preparations may be added to augment biologic dressings, which are commercially available from a variety of sources, with stem cells and growth factors to treat burns, skin pressure ulcers, other chronic open wounds, corneal ulcers, and as a dressing following corneal transplant and other ocular procedures. In some embodiments, amniotic fluid-derived preparations may be used as a component of the extracellular matrix in bioengineered connective tissue scaffolding for tissue and organogenesis using extraembryonic stem cells and other progenitor cells. Amniotic fluid-derived preparations may possess the anti-inflammatory properties of AF, and in some embodiments, amniotic fluid-derived preparations of the invention may be used to prevent the development of postoperative adhesions between the tendon, tendon sheath, and associated tissue following tenolysis, synoviolysis, surgical repair of a damaged tendon, and surgical debridement of necrotic or damaged tendon tissue. Amniotic fluid-derived preparations may also be useful to prevent nerve cell death and promote axonal regeneration following early repair of peripheral nerve transections. Therefore, in some embodiments, amniotic fluid-derived preparations may be used in a method to prevent nerve cell death and/or to promote axonal regeneration following early repair of peripheral nerve transections.

An injectable amniotic fluid-derived preparation allows for use of the composition in both surgical and minimally invasive settings. The injectable amniotic fluid-derived preparation may be injected into a defined closed space near the end of the surgical procedure, but prior to closing superficial layers of muscle, fascia, and skin at a time when precise placement of the preparation under the surgeon's direct visualization is possible. For example, an injectable amniotic fluid-derived preparation, depending on the viscosity of the final product, is delivered by injection though a hypodermic needle as small as 30-gauge (“G”) into a closed tendon sheath following tenolysis or tendon repair, into a closed joint capsule following repair of intra-articular cartilage, ligaments, or total joint replacement, into the peritoneal cavity following closure of the abdominal wall, into the pleural space following closure of the chest wall, and into the subdural space following closure of the spinal or intracranial dura mater. An injectable amniotic fluid-derived preparation of higher viscosity is injected through a 23G, 22G, 21G, 20G, 18G, 16G, or larger-bore hypodermic needle in these and other surgical and minimally invasive applications. An injectable amniotic fluid-derived preparation of lower viscosity is injected through a 25G or 30G needle for use in fine neural repair, aesthetic surgery, and other applications. Following wound closure, an injectable amniotic fluid-derived preparation may also be re-injected into the defined closed space during the perioperative and postoperative period if deemed useful by the surgeon or other healthcare provider.

An injectable amniotic fluid-derived preparation may also be injected into a tissue bed in a minimally invasive non-surgical setting. For example, a syringe containing a quantity of the amniotic fluid-derived preparation is fitted with a hypodermic needle of suitable size for the intended application. The needle is directed to the target tissue bed using visualization and palpation of external landmarks by the provider. Placement of the needle within the target tissue space or tissue may be facilitated with fluoroscopy or other non-invasive imaging modalities. Some example minimally invasive uses of amniotic fluid-derived preparations include intra-articular injection for treatment of injured ligaments, cartilage, and bone; intra-capsular injection of tendon injuries, synovitis, tenosynovitis, and other inflammatory joint conditions; intra-thecal injection for treatment of spinal cord and brain injuries, aseptic meningitis, and other central neurological infections and inflammatory conditions; and other minimally invasive non-surgical applications.

In all of these and other applications, there is strong evidence that the presence of active biomolecules in the amniotic fluid-derived preparations improves healing across a broad range of tissue types, locations within the body, and clinical conditions. Reporting of clinical results may eventually lead to the use of amniotic fluid-derived preparations as a standard therapy and possibly even the best practice for the treatment of a variety of conditions. Results reporting requires laboratory experimentation and human clinical trials to generate data for review and interpretation in light of currently available practices and results therefrom. Meaningful interpretation of these generated data, however, depends on reproducibility. Reproducibility requires standardization of materials and techniques. Standardization of amniotic fluid-derived preparations should include a viable cell count per volume and the biologic activity of one or more specific proteins or other biologically active molecules present in the amniotic fluid-derived preparation. In AF collected from individual donors, substantial differences in both the absolute amount and biologic activity per unit volume of proteins and other biologically active molecules in the final preparation will exist based upon the gestational age at collection, other maternal and fetal factors, and preparation methods used.

Preparation and sterilization of an amniotic fluid-derived preparation for later use typically includes packaging, sterilization, lyophilization (in some embodiments), and storage. Lyophilization helps maintain sterility during storage by discouraging microbial growth. Lyophilization additionally facilitates standardization of the final amniotic fluid-derived preparation in terms of biologic activity per unit volume of the amniotic fluid-derived preparation under standardized parameters. Lyophilization may be accomplished by freezing under controlled conditions to minimize water-ice crystal formation and cellular disruption in products wherein preservation of cell viability is desired. Preservation of viable stem cells is not currently possible with lyophilization. It is not fully known how drying and storage affect the concentration of the biologically active non-cellular components of AF, though a significant decrease in concentration of intact proteins and other large biomolecules is possible. Sterilization by heat or radiation destroys the cellular components of AF, including stem cells. Thermal or irradiative sterilization methods may also denature proteins and alter or destroy other large biologically active molecules. Some amniotic fluid-derived preparations partially reconstitute the concentrated cellular component using a buffered, balanced electrolyte tissue preservative solution prior to packaging and storage.

What is lacking in the prior art, therefore, is an amniotic fluid-derived preparation incorporating an effective concentration of cellular and biomolecular products from an individual donor within the largest possible pool of volunteer donors with a standardized biological activity and potency, packaged and stored to preserve cellular viability and biological activity of the preparation.

Embodiments of this invention address these and other fundamental requirements of an amniotic fluid-derived preparation—high concentrations of beneficial biomolecules and viable cells in a standardized preparation with reproducible biologic effects which are preserved throughout packaging, frozen storage, and thawing; essentially no feto-maternal antigenic material, and minimal waste of available donor AF. The amniotic fluid-derived preparation comprises AF which has been separated into its cellular and non-cellular elements, washed and assayed, concentrated with regard to the cellular component, a protein fraction, or both; and then reconstituted with an acceptable fluid to preserve cell viability and biologic activity throughout packaging, freezing, and storage.

Disclosed is an amniotic fluid-derived preparation comprising a protein fraction, a cell type, and a fluid. Some embodiments of the invention comprise additional compounds and characteristics to standardize the biologic effects of the amniotic fluid-derived preparation and to preserve cell viability and protein activity following freezing, storage, and thawing. The amniotic fluid-derived preparation may be used by medical providers as an injectable fluid or non-injectable gel preparation, either by intraoperative application or injection, non-operative percutaneous injection, or direct application to injured, ischemic, infected, or otherwise damaged tissue. The amniotic fluid-derived preparation may also be used by laboratory researchers as a reproducible source of standardized material for basic science research on the effects of AF preparations on healthy, diseased, and damaged tissue in the field of regenerative medicine, orthopedics, neurology, neurosurgery, gynecologic surgery, and in other clinical, basic medical science, and related scientific disciplines. Use of a reconstituted amniotic fluid-derived preparation comprising biocompatible fluids such as an isotonically balanced buffered electrolyte solution and/or a cryopreservative maximizes delivery of a wide range of regenerative and similarly beneficial biologic substances within a non-antigenic liquid or gel preparation to the targeted treatment tissue.

In some embodiments, the amniotic fluid derivative further comprises a concentrated exosome component. Concentrated exosome components may include major histocompatibility complex class I or II molecules, cytosolic chaperone proteins, microRNAs (for example, miR-150, miR-142-3p, miR-451, miR-15b, miR-16, miR-196, miR-21, miR-26a, miR-27a, miR-92, miR-93, miR-320, miR-20, let-7a, miR-146a, let-7f, miR-20b, miR-30e-3p, miR-222, miR-6087, miR-126, miR-130a, miR-135b, miR-200a, miR-200b, miR-200c, miR-203, miR-205, miR-141, miR-155, miR-17-3p, miR-106a, miR-146, miR155, miR-191, miR-192, miR-212, miR-214, and miR-210), Rab GTPase. SNAREs, flotillin, subunits of trimeric G proteins, cytoskeletal proteins, annexins, integrins, cholesterol, sphingomyelin, ceramides, hexosylceramides, phosphatidylcholine, phosphatidylethanolamine, phosphatidylserine, elongation factors, Delta 4, syndecan-1, STATS, PDGF, VEGF, hepatocyte growth factor, sonic hedgehog (SHH), MFGE8, GW182, AGO2, Hsp60, Hsc70, Hsp90, Hsp20, 14-3-3 epsilon, PKM2, nuclear factor κB (NFκB), tetraspanins, CD9, CD63, CD80, CD86, CD19, CD81, CD82, CD53, CD37, CD34, CD41, CD62p, TSG101, matrix metalloproteinases (MMPs), extracellular matrix metalloproteinase inducer, AU rich element binding proteins (e.g., KRSP and TTP), RNA binding proteins (e.g., MPP6 and C1D), Rrp40, hCsl4, hRrp4, hRrp40, PM/Scl-75, Dis3, Dis3L1, Rrp44-H1, Rrp44-H2, Rrp44-H3, hRrp41, hRrp42, hMtr3, hRrp43/OIP2, hRrp46, and PM/Scl-100, or any combination thereof.

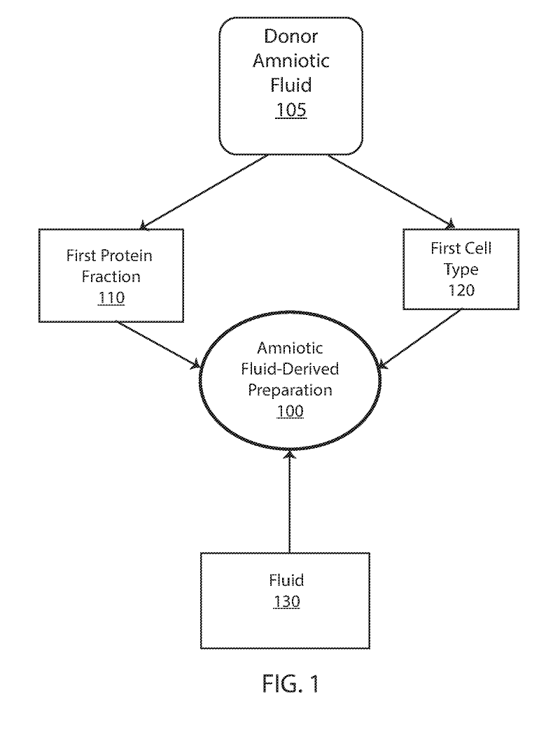

FIG. 1 is a schematic diagram of an amniotic fluid-derived preparation 100. Amniotic fluid-derived preparation 100 comprises a first protein fraction 110, a first cell type 120, and a fluid 130. In some embodiments, a donor amniotic fluid 105 comprises first protein fraction 110 and first cell type 120. As shown in FIG. 1, arrows indicate donor amniotic fluid 105 is separated into first protein fraction 110 and first cell type 120, which are combined with fluid 130 to form amniotic fluid-derived preparation 100.

First protein fraction 110 and first cell type 120, in some embodiments, are formed following collection and centrifugation of donor amniotic fluid 105. In some embodiments, donor amniotic fluid 105 is collected from a volunteer human donor. Accepting AF from volunteer donors and excluding any non-volunteer and paid donors from the donor pool is consistent with internationally well-established tissue donation protocols by reducing the risk of donor-transmitted infection to a recipient of amniotic fluid-derived preparation 100. Screening of potential volunteer donors, therefore, includes obtaining a comprehensive past medical and social history, complete blood count, liver and metabolic profile, and serologic testing for HBV, HCV, HIV, and other infectious agents, in some embodiments.

In some embodiments, donor amniotic fluid 105 comprises AF collected from a non-human donor animal. A lack of expression of HLA-1 and HLA-D related (“HLA-DR”) epitopes makes cross-species use of amniotic fluid-derived preparations possible. In some embodiments, amniotic fluid-derived preparation 100 comprises donor amniotic fluid 105 from a non-human donor which is completely de-cellularized by processing prior to combination with first cell type 120 and fluid 130. For example, in some embodiments, AF from a non-human donor animal (for example, a non-human mammal, for example, a primate) is placed in a centrifuge at 400 g for ten (10) minutes and the resulting supernatant is free of cells and cellular debris. In a second non-limiting example, the AF from a non-human donor animal is filtered through a filter with a 0.22 micrometer pore size, wherein all cells and cellular debris are removed from donor amniotic fluid 105.

In some embodiments, donor amniotic fluid 105 is collected during delivery by Cesarean section. The use of a Cesarean-obtained donor amniotic fluid 105 to prepare amniotic fluid-derived preparation 100 is preferable in some embodiments because donor amniotic fluid 105 collected by Cesarean section is obtained and packaged under strict sterile technique in the operating room, with essentially no microbial contamination. In some embodiments, donor amniotic fluid 105 is collected into a sterile suction canister liner, following surgical exposure of the intact fetal membranes through a trans-abdominal incision and uterine myotomy, by the surgeon-obstetrician nicking the amniotic membrane and inserting a suction catheter tip into the semi-transparent placental sac under direct vision so as to prevent injury to the infant. Following collection of donor amniotic fluid 105, which takes approximately five to ten seconds, the baby is delivered by the surgeon-obstetrician. Operating room personnel familiar with sterile technique and tissue handling perform all steps necessary to prepare donor amniotic fluid 105 for packaging.

In some embodiments, the sterile container containing donor amniotic fluid 105 collected under sterile conditions in the operating room is securely closed and placed in a donor tissue specimen bag. This first specimen bag is then placed within a second bag, which is sealed, labeled, and taken from the operating room for packaging on an ice bath in an insulated container. A patient data sheet containing information regarding the maternal donor is placed in the container, and a separate copy of this information is recorded and logged prior to closing the package. The packaged specimen container is then immediately transported to a processing facility by staff who rotate on call, such that there is minimal delay following delivery before the donor tissue arrives at the separate facility for processing.

Despite the preference for a Cesarean-collected donor amniotic fluid 105, trans-vaginally collected AF is utilized in some embodiments to increase the pool of potential donors. In some embodiments, trans-vaginal collection of AF is performed in a clinical setting wherein trans-vaginal rupture of fetal membranes is indicated to initiate or promote the progression of labor. Similar sterile collection and handling practices as discussed herein above are utilized, although donor amniotic fluid 105 is collected with a sterile suction cannula placed through the dilated cervix against the intact fetal membranes prior to rupturing the fetal membranes with an amnion hook or similar instrument. Great care must be afforded the trans-vaginally-collected donor amniotic fluid 105 to prevent microbial contamination. Trans-vaginally-collected AF is not an acceptable donor amniotic fluid 105 if there is fecal, blood, or other grossly visible contamination noted in the AF or in proximity to the vagina at the time of collection. Neither a trans-vaginally-collected donor amniotic fluid 105 nor a Cesarean-collected donor amniotic fluid 105 is acceptable to form amniotic fluid-derived preparation 100 if meconium is present in the AF or if there is any visible meconium discoloration or staining of the AF.

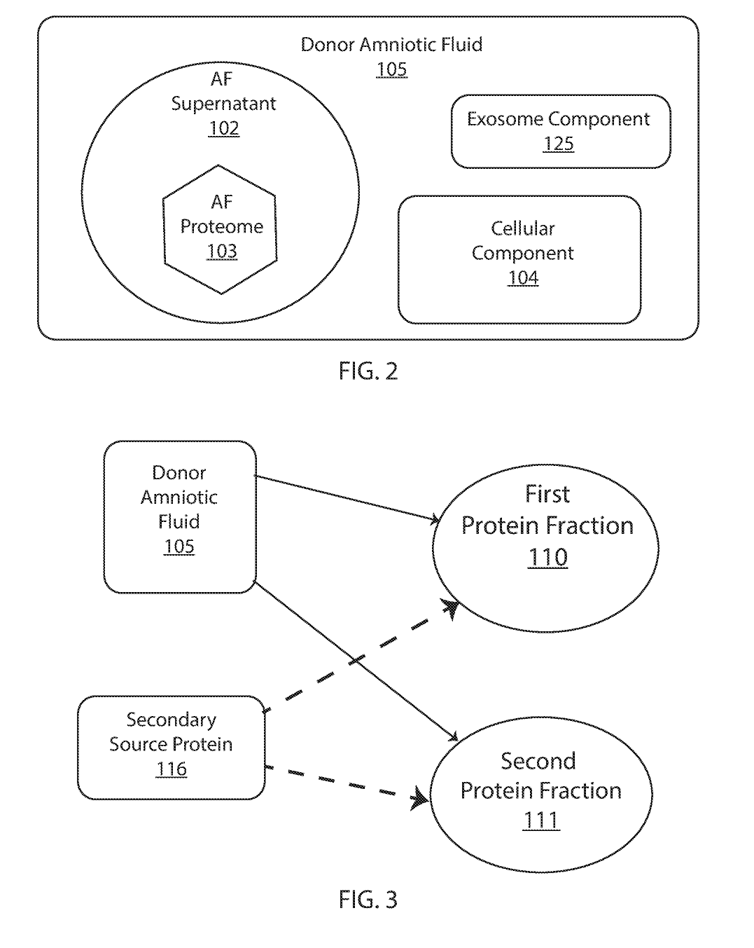

FIG. 2 is a schematic representation of the constituent components of donor amniotic fluid 105. As shown in FIG. 2, donor amniotic fluid comprises an AF supernatant 102, an exosome component 125, and a cellular component 104. The water-based AF supernatant 102, in turn, comprises an AF proteome 103 and a variety of other substances (not shown in FIG. 2), including electrolytes, phospholipids, carbohydrates, and urea. AF proteome 103 is the entire set of products of transcription manifest as proteins and polypeptides within donor amniotic fluid 105, the composition of which will vary between individual donor amniotic fluids 105.

In some embodiments, the non-cellular components of AF, including AF proteome 103, are separated from cellular component 104 by centrifugation using commercially available equipment and established techniques known to those in the art. For example, in some embodiments, the donor amniotic fluid 105 is centrifuged at a relative centrifugal force (“RCF”) of between about 300 g and about 500 g for ten (10) minutes. At this speed and duration, the supernatant is essentially cell free, with all cells and cellular debris from donor amniotic fluid 105 present in the pellet. Other non-limiting examples include RCFs from 300 g to 1000 g for a duration of about from three (3) to about ten (10) minutes. In some embodiments, amniotic fluid 105 is centrifuged at an RCF of less than about 300 g for between about five (5) and about ten (10) minutes. In some embodiments, amniotic fluid 105 is centrifuged at an RCF greater than about 1000 g. In some embodiments, amniotic fluid 105 is centrifuged for a duration of greater than ten about (10) minutes. The choice of speed and duration of AF centrifugation will depend upon factors such as the mechanical fragility characteristics of specific cells retained as viable cells, proteins, and other large molecule substances to be preserved for use in amniotic fluid-derived preparation 100. In various embodiments, the donor amniotic fluid may be centrifuged at about 100 g, about 200 g, about 300 g, about 400 g, about 500 g, about 600 g, about 700 g, about 800 g, about 900 g, about 1000 g, between about 100 g and 300 g, between about 300 g and about 500 g, between about 500 g and about 700 g, between about 700 g and about 900 g, or between about 800 g and about 1000 g. In some embodiments, the donor amniotic fluid may be centrifuged for about 2 minutes, about 10 minutes, about 20 minutes, about 30 minutes, about 40 minutes, about 50 minutes, about 1 hour, about 2 hours, between about 2 minutes and about 5 minutes, between about 5 minutes and about 10 minutes, between about 10 minutes and about 30 minutes, between about 30 minutes and about 1 hour, or between about 1 hour and about 2 hours. In some embodiments, the donor amniotic fluid may be centrifuged at about 4° C., at about 25° C., or at about 37° C.

Following centrifugation, the water-based AF supernatant 102 comprises AF proteome 103 and a variety of other substances not shown in the figures, including, but not limited to, electrolytes, phospholipids, carbohydrates, and urea. Depending upon the intended specific therapeutic use of amniotic fluid derived preparation 100, it may be desirable for amniotic fluid-derived preparation 100 to be depleted of one or more components of the group of components comprising individual electrolytes, phospholipids, carbohydrates, urea, and the like present in AF supernatant 102. For example, amniotic fluid derived preparation 100 with first protein fraction 110 comprising vascular endothelial growth factor is depleted of phospholipids and urea, in some embodiments. Consequently, a protein isolation and concentration method is used, in some embodiments, to form first protein fraction 110. Depletion of first protein fraction 100 of such components found in AF supernatant 102 is accomplished by a variety of methods. In some embodiments, first protein fraction 100 is depleted of AF supernatant 102 components by isolating an individual specific protein or a plurality of proteins using one of the methods described herein below. Dialysis against a solution of a defined composition is a relatively simple and efficient method to deplete or otherwise manipulate the concentrations of individual components comprising AF supernatant 102 to form first protein fraction 110. This is a non-limiting example; other techniques, such as chromatography, may also be used.

First protein fraction 110, in some embodiments, is isolated from AF proteome 103. The normal human AF proteome comprises over one hundred and thirty (130) separate proteins (Tsangaris, et al. (2006) “The Normal Human Amniotic Fluid Supernatant Proteome” In Vivo 20:279-90.). The individual proteomic proteins comprise products of maternal and fetal transcription, and may vary somewhat depending upon the gestational age of the fetus, the gender of the fetus, and any existing fetal or maternal chromosomal or metabolic abnormalities. First protein fraction 110 is any one protein or plurality of proteins in any number or combination. In some embodiments, for example, first protein fraction 110 is the complete AF proteome 103. In some embodiments, a portion of the water and other non-protein constituent compounds of AF supernatant 102 are removed wherein the overall proteomic concentration is increased (concentrated). Multiple procedures are available and known in the art to separate, isolate, and concentrate proteins from complex biological fluids, such as AF supernatant 102. Some non-limiting examples of techniques utilized in concentration of AF proteome 103 of AF supernatant 102 include precipitation, precipitation with centrifugation, precipitation with filtration, continuous or discontinuous density gradient centrifugation, protein electrophoresis, and the like. These non-limiting examples also apply to concentrating the overall protein concentration of first protein fraction 110. Such techniques may alter the composition and biologic activity of individual protein constituents of AF proteome 103.

For example, in some embodiments, any one or more than one of vascular endothelia growth factor (“VEGF”), epidermal growth factor (“EGF”), endocrine gland-derived vascular endothelial growth factor (“EG-VEGF”), hepatocyte growth factor (“HGF”), erythropoietin (“EPO”), platelet-derived growth factor (“PDGF”), monocyte chemoattractant protein 1 (“MCP1”), stromal cell-derived factor (“SDF”), angiogenin (“ANG”), angiopoietin, fibroblast growth factor (“FGF”), insulin-like growth factor (“IGF”), insulin-like growth factor binding protein (“IGFBP”), matrix metalloproteinases (“MMPs”), the enzyme hyaluronidase, or tissue inhibitor of metalloproteinases (“TIMP”) are concentrated in first protein fraction 110, second protein fraction 111, or first protein fraction 110 and second protein fraction 111.

In some embodiments, first protein fraction 110 is a product of precipitation of AF supernatant 102. In some embodiments, precipitation of AF supernatant proteome 103 is performed using ammonium chloride according to protocols known in the art. The use of ammonium chloride is by way of example only; any suitable salt and specific precipitation technique known in the art may be employed. Following precipitation, the treated AF supernatant comprising the precipitated AF proteomic protein component is centrifuged at a selected RCF and for a duration sufficient to separate the protein-containing precipitate, sometimes referred to as the “pellet,” and the supernatant. The resulting new supernatant comprising water, lipids, carbohydrates, phospholipids, and other constituents is removed from the protein-containing pellet or precipitate. In some embodiments, the pellet or precipitate is washed by performing one or more cycles of re-suspending in buffer solution and re-centrifuging. In some embodiments, the salt, whether ammonium sulfate or other salt used for precipitation, is removed by dialysis or other suitable technique known in the art. The precipitate is combined with a minimal volume of an appropriate buffer solution to form a solution of first protein fraction 110. The choice of buffer, both for washing the precipitate and storing first protein fraction 110, is chosen to maintain pH within a range based upon the functional structure and physiochemical properties of first protein fraction 110, such as the isoelectric point and other physiochemical characteristics of the protein or proteins comprising first protein fraction 110. Some non-limiting examples of buffers include solutions of chloride (hydrochloric acid) salts of potassium, glycine, aconitate, citrate, acetate, citrate-phosphate, succinate, phthalate-sodium hydroxide, maleate, phosphate, boric acid, 1-amino-2methyl-1,3-propanediol, glycine-sodium hydroxide, borax-sodium hydroxide, carbonate-bicarbonate, and the like.

Conversely, in some embodiments, first protein fraction 110 comprises supernatant proteome 103 depleted of one or more constituent proteins by precipitation. Otherwise stated, rather than incorporating the precipitated protein or proteins into first protein fraction 110, the precipitated protein or proteins are removed from supernatant proteome 103, leaving the remaining constituent proteins of supernatant proteome 103 as comprising first protein fraction 110. In some embodiments, the depleted protein is albumin. In some embodiments, the depleted protein is an immunoglobulin, for example, IgG, IgM, IgA, IgD, or IgE.

In some embodiments, an individual protein or group of proteins comprising first protein fraction 110 is separated from the remainder of the AF supernatant proteome using a density-gradient centrifugation technique. In one non-limiting example protocol, 100 microliters (0.1 milliliters) of AF supernatant 102 is layered onto a sucrose gradient solution in a centrifuge tube, the gradient comprising (from bottom of the tube to the liquid surface) 950 microliters of 40% sucrose solution; 950 microliters of 31.25% sucrose solution; 950 microliters of 22.5% sucrose solution; 950 microliters of 13.75% sucrose solution; and 950 microliters of 5% sucrose solution. The sucrose gradient should be refrigerated at 4° C. for twelve (12) to sixteen (16) hours to allow a linear gradient to form prior to layering AF supernatant 102 and centrifuging. The tube is then centrifuged at approximately 237,000 g for four (4) hours. The tube is removed from the centrifuge and placed in an ice bath. In some embodiments, the protein fractions within a microcentrifuge tube are precipitated by adding 300 microliters (an equal volume) of trichloroacetic acid to the microcentrifuge tube containing the protein fraction and the tube is placed on ice for thirty (30) minutes. The tube is then centrifuged at 15,000 g for fifteen minutes at 4° C. The supernatant is separated, such as by pipetting or decanting techniques. The protein pellet is re-suspended in 100 microliters of buffered electrolyte solution, such as PBS, for example. In this example, and some other embodiments, first protein fraction 110 comprises the resulting re-suspended protein pellet suspension.

Precipitation using ammonium chloride or other suitable compound to isolate and concentrate first protein fraction 110 is by way of example only. Other methods known and practiced in the art, such as liquid chromatography, ultrafiltration-centrifugation, ligand-antibody affinity binding with magnetic separation, and the like may be utilized. The choice of method and details of the procedure wherein first protein fraction 110 is formed are determined by the physiochemical and immunologic characteristics of the specific protein or group of proteins comprising first protein fraction 110.

Additional quantities of first protein fraction 110 from an individual donor are produced, in some embodiments, by extracting constituent intracellular protein(s) from a cellular component of AF. AF from which intracellular proteins are extracted may be donor AF 105, in some embodiments. In some embodiments, intracellular proteins are extracted from AF collected from a separate donor. In some embodiments, for example, cellular component 104 comprising the cellular “pellet” is “washed” by re-suspending the pellet in a buffer solution followed by re-centrifugation and removal of the supernatant comprising the buffer solution one or more times. The washed cellular component 104 pellet is re-suspended in a quantity of buffer solution to form a cellular suspension in buffer of cellular component 104. An aliquot of this suspension is removed and the cells in the aliquot are disrupted by using an established technique known in the art, releasing high concentrations intracellular proteins into the suspension. Non-limiting examples of such techniques include serial freezing-and-thawing, use of detergents, sonication, high pressure filtration, or treatment with organic solvents to disrupt the cell membrane releasing membrane receptors and other membrane proteins.

For example, in a particular embodiment, an aliquot of the cellular suspension is further washed through two suspension/centrifugation cycles with phosphate buffered saline (“PBS”) and the washed cells are placed in culture dishes, on ice. To each dish is added 1.0 milliliter of a detergent lysis buffer, such as a 0.01%-0.05% aqueous solution of sodium dodecyl sulphate or NP-40. A commercially available lytic reagent, such as Mammalian Protein Extraction Reagent (“M-PER”) available from Thermo Fisher Scientific of Waltham, Mass., for example, may also be used. The cells are then incubated on ice for between ten (10) and thirty (30) minutes, periodically rocking the dishes gently. A dish is then tilted slightly on the ice bed to allow the buffer solution containing the cellular lysate to drain to one side, where it is removed with a pipette. The pipetted lysate is centrifuged at 20,000 g for ten (10) minutes at 4° C. The supernatant is carefully removed to a fresh centrifuge tube, taking care not to disturb the debris pellet. The lysate may be stored on ice, or flash-frozen using a dry ice/ethanol mixture and then stored at minus seventy degrees Celsius (−70° C.).

Alternatively, after cellular disruption, proteins are extracted and purified from the resulting cellular lysate by use of another aforementioned technique under protocols known in the art; non-limiting examples including precipitation, immunoprecipitation, centrifugation on a sucrose, Percoll®, or alternative density gradient; protein electrophoresis; chromatography; fluorescent or magnetic bead-based immunoaffinity separation; other aforementioned non-limiting examples, and the like; in some embodiments.

The resulting purified protein component or specific protein(s) are then added to first protein fraction 110 or second protein fraction 111. In some embodiments, first protein fraction 110 comprises a growth factor. Example growth factors found in AF supernatant comprising first protein fraction 110 include VEGF, HGF, angiopoietin, PDGF, and FGF. Some embodiments of amniotic fluid-derived preparation 100 wherein first protein fraction 110 comprises any of these five examples of growth factors are for use in clinical situations wherein de novo induction of vasculature ingrowth resulting in tissue neovascularization through the bioactivity of the growth factor(s) is sought. Non-limiting examples of such situations include healing of wounds in chronically ischemic tissue, such as hypo-perfused tissue or irradiated tissue; incorporation of surgically placed cadaver bone grafts; pedicle flap grafts, free tissue flaps, and the like.

In some embodiments, first protein fraction 110 comprises one or more signaling ligands. Some non-limiting examples of signaling ligands found in AF supernatant comprising first protein fraction 110 include MCP1, stromal cell derived factor one (“SCDF1”), and stem cell factor (“SCF”). These three example signaling ligand proteins are all intrinsic to human AF. Some embodiments of amniotic fluid-derived preparation 100 wherein first protein fraction 110 comprises any of these three examples of signaling ligand proteins are for use in clinical situations wherein regulation and trafficking of host-derived mesenchymal stem cells is desirable, such as healing of injured cartilage, hepatocellular regeneration, incorporation of a surgically placed cadaver bone graft, incorporation of a surgically placed tissue scaffold, and the like.

In some embodiments, first protein fraction 110 comprises MMPs and TIMPs. The balance between MMPs and TIMPs is partially responsible for mediating the degradation of collagens and other salient components of the extracellular matrix during the development of tendon pathology. In a systematic survey of the transcriptomics and proteomics associated with the molecular pathogenesis of human tendinopathies of the rotator cuff and biceps, significant increases were observed in the expression of collagen I, collagen III, MMP 1/9/13, and TIMP1 as well as a decrease in MMP3 (Del Bueno, et al., (2012) “Metalloproteases and rotator cuff disease” J Shoulder Elbow Surg. 21:200-08). Accordingly, in some embodiments, wherein first protein fraction 110 of comprises MMPs and TIMPs, amniotic fluid-derived preparation 100 is used in clinical situations wherein remodeling of the extracellular matrix, such as healing of tendon damage, requires a reduction and reversal of continued pathologic tissue degradation.

Additionally, hyaluronic acid (“HA”) present in AF supernatant 102 and first protein fraction 110, in some embodiments, comprises a demonstrated pro-regenerative bioactivity. Such regenerative activity allows for remodeling of the extracellular matrix and facilitates healing. For example, the absence of scarring and fibrosis during healing of fetal skin lesions has been directly correlated to the extended presence of HA in amniotic fluid during gestation (Mast, et al., (1992) “Scarless wound healing in the mammalian fetus” Surg Gynecol Obstet 174:441-51; West, et al., (1997) “Fibrotic healing of adult and late gestational fetal wounds correlates with increased hyaluronidase activity and removal of hyaluronan” Int J Biochem Cell Biol 29:201-10). Factors present in AF, and consequently AF supernatant 102 and first protein fraction 110, that specifically stimulate the production of HA and therefore facilitate the tissue regeneration process have also been described (Longaker, et al., (1990) “Studies in fetal wound healing, VII. Fetal sound healing may be modulated by hyaluronic acie stimulating activity in amniotic fluid” J Pediatr Surg 25:430-33). Factors present in human AF, and therefore AF supernatant 102 and first protein fraction 10, in some embodiments, have been demonstrated to modulate the activity of critical proteases functional during the regenerative process, including collagenases, hyaluronidases, elastases, and cathepsin B (Gao, et al., (1994) “Effects of amniotic fluid on proteases: a possible role of amniotic fluid in fetal sound healing” Ann Plast Surg 33:128-34). Additionally, the presence of HA and HA-stimulating bioactivity in human AF has been linked to observed neochondrogenesis in rabbit models of perichondrial grafting (Ozgenel, et al., (2004) “Effects of human amniotic fluid on cartilage regeneration from free perichondrial grafts in rabbits” Br J Plast Surg 57:423-28; Kavakli, et al., (2011) “Effects of human amniotic fluid on costal cartilage regeneration (an experimental study)” Thorac Cardiovasc Surg 59:484-89).

In some embodiments, first protein fraction 110 comprises a receptor molecule antagonist. For example, the interleukin-1 (“IL-1”) receptor agonist has been identified in human AF (Silini, et al., (2013) “Soluble factors of amnion-derived cells in treatment of inflammatory and fibrotic pathologies” Curr Stem Cell Res & Therapy 8:6-14). IL-1 receptor antagonist is a potent anti-inflammatory cytokine present in AF, and consequently AF supernatant 102 and first protein fraction 110, in some embodiments.

In some embodiments, first protein fraction 110 comprises a cytokine. In addition to the IL-1 receptor antagonist, human AF also comprises interleukin 10 and prostaglandin E2 (“PGE2”), all of which are potent anti-inflammatory cytokines (ibid).

In some embodiments, first protein fraction 110 comprises a transcriptional regulator. For example, in some embodiments, first protein fraction 110 comprises octamer-binding transcription factor 4 (“OCT4”).

In some embodiments, first protein fraction 110 comprises an immune regulator. For example, in some embodiments, first protein fraction 110 comprises transforming growth factor beta (“TGF-β”). Embodiments of amniotic fluid-derived preparation 100 wherein first protein fraction 110 comprises TGF-β can be used clinically to blunt the immune response through TGF-β's known actions inhibiting lymphoid cells, including secretion of cytokines such as interleukin I, interleukin II, and tumor necrosis factor alpha from T lymphocytes; and suppressing differentiation and antibody secretion of B lymphocytes while augmenting the myloid immune response by acting as a chemoattractant for macrophages and monocytes.

In some embodiments, first protein fraction 110 comprises VEGF. In some embodiments, first protein fraction 110 comprises human growth hormone (“HGH”). In some embodiments, first protein fraction 110 comprises EPO. In some embodiments, first protein fraction 110 comprises TPA. In some embodiments, first protein fraction 110 comprises angiogenin. In some embodiments, first protein fraction 110 comprises angiopoietin. In some embodiments, first protein fraction 110 comprises PDGF. In some embodiments, first protein fraction 110 comprises EGF. In some embodiments, first protein fraction 110 comprises basic fibroblast growth factor. In some embodiments, first protein fraction 110 comprises fibroblast growth factor 4. In some embodiments, first protein fraction 110 comprises monocyte chemoattractant protein. In some embodiments, first protein fraction 110 comprises stromal cell derived factor 1. In some embodiments, first protein fraction 110 comprises stem cell factor. In some embodiments, first protein fraction 110 comprises a MMP. In some embodiments, first protein fraction 110 comprises a TIMP. In some embodiments, first protein fraction 110 comprises interleukin. In some embodiments, first protein fraction 110 comprises interleukin 10. In some embodiments, first protein fraction 110 comprises prostaglandin E2.

In some embodiments, first protein fraction 110 comprises a secondary source protein 116. The secondary source protein may be present in the AF proteome but derived from a non-AF source, such as a genetically recombinant bacterium, yeast, human tissue cultured cells, and the like. Examples of proteins available through non-AF sources include VEGF, HGH, EPO, and tissue plasminogen activator (“TPA”). Alternatively, the secondary source protein may not be present in the AF proteome and derived from a non-AF source.

FIG. 3 is a schematic representation of the constituent components of first protein fraction 110 and a second protein fraction 111. In some embodiments, amniotic fluid-derived preparation 100 comprises a second protein fraction 111. In some embodiments, second protein fraction 111 comprises an AF proteome 103-constituent protein. In some embodiments, second protein fraction 111 comprises secondary source protein 116.

AF supernatant 102 additionally comprises non-cellular elements, including exosomes, cell free fetal DNA (“cffDNA”), and the like. In some embodiments, amniotic fluid-derived preparation 100 comprises exosome component 125. Exosomes present in AF comprise fetal-derived exosomes which contain immune-modulatory and anti-inflammatory proteins in addition to cffDNA. In a non-limiting example, a concentrated exosome component is formed by centrifugation of donor amniotic fluid 105 on a density gradient, such as a sucrose gradient or a Percoll® gradient, using standard techniques known in the art. Exosome component 125 has an exosome concentration of greater than thirty (30) micrograms per milliliter. In some embodiments, AF supernatant 102 or cellular component 104 are substantially depleted of exosomes by removing the exosome-bearing density fraction following a separation method, such as density gradient centrifugation, for example. The aforementioned techniques for either concentrating or substantially removing exosome component 125 from donor amniotic fluid 105 are by way of example only; other suitable techniques may be used.

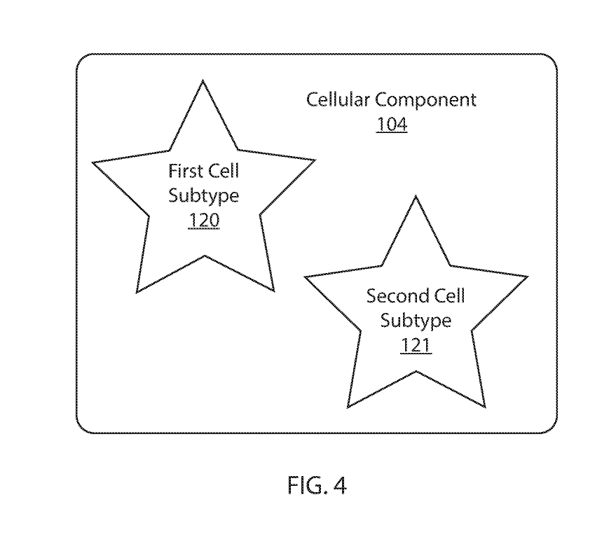

FIG. 4 is a schematic representation of the constituent components of cellular component 104. In some embodiments, cellular component 104 comprises a first cell type 120. Cellular component 104, in some embodiments, comprises first cell type 120 and a second cell type 121. Upon collection but prior to processing, donor AF 105 comprises many different cell types. These constituent cell types may be generally divided into two groups: 1) progenitor cells; and 2) differentiated cells. The progenitor cell component may be further divided into a pluripotent cell group and a committed cell group. Pluripotent cells retain the ability to differentiate into any germ line; i.e. endodermal, mesodermal, or ectodermal-derived tissues. Committed progenitor cells will differentiate into defined germ cell lines or organ-specific cell types.

In some embodiments, cellular component 104 comprises first cell type 120 without removal or addition of cell subtypes. Following initial separation of donor amniotic fluid 105 into AF supernatant 102 and cellular component 104, cellular component 104 is “washed,” in some embodiments, by multiple cycles of re-suspension of cellular component 104 in buffer solution and by re-centrifugation (or alternative separation technique) with removal of the supernatant comprising the buffer solution.

Conversely, cellular component 104, in some embodiments, is separated into groups of constituent cell subtypes which are isolated using various techniques known in the art and concentrated in amniotic fluid-derivative preparation 100. Some non-limiting examples of these cell types, comprising first cell type 120 and second cell type 121, include cells bearing surface receptors identifying the cell as a mesenchymal stem cell, a progenitor cell, an epithelial cell, such as a cell expressing surface receptor CD44, a cell expressing surface receptor CD29, a cell expressing surface receptor CD49e, a cell expressing surface receptor CD54, a cell expressing surface receptor CD44, a cell expressing surface receptor CD326, a cell expressing surface receptor CD166, a cell expressing surface receptor CD271, a cell expressing surface receptor CD45, a cell expressing surface receptor CD349, and a cell expressing surface receptor CD140b, in some embodiments.

In some embodiments, cellular component 104 comprises a cellular component substantially depleted of epithelial cells, mesenchymal cells, or of any of the aforementioned cells bearing cell surface receptors identified by non-limiting example in the preceding paragraph.

Some non-limiting examples of cell separation techniques include density-gradient centrifugation, magnet-activated cell sorting (“MACS”) utilizing polymer-bound monoclonal antibodies to cell surface receptors, other antibody-based techniques such as florescent antibody-bonded colloidal bead separation, for example; microfluidic techniques, and the like.

In some embodiments, density-gradient centrifugation within a sucrose solution or a colloidal silica suspension, such as Percoll®, for example, is employed to separate the heterogeneous cell populations comprising cellular component 104 into a number of subpopulations based upon the buoyant density of the subtype. During centrifugation, cells will “band” on the gradient in levels corresponding to the relative buoyant density of each subpopulation. The region containing the desired subpopulation to comprise first cell type 120 is removed from the banded supernatant. Conversely, a region not comprising first cell type 120 is removed, in some embodiments. A region not comprising first cell type 120 or second cell type 121 may be a region comprising dead cells.

In some embodiments, viability testing of first cell type 120 separated from cellular component 104 is conducted to quantify viable cells comprising first cell type 120. In some embodiments, viability testing comprises a standard dye exclusion technique, such as Trypan blue exclusion by “live-dead staining,” known and established in the art is used. An alternative dye exclusion assay, such as a calcein assay or an ethidium bromide is used, in some embodiments. In some embodiments, viability testing of second cell type 121 is performed. In some embodiments, viability testing is performed on second cell type 121.

In some embodiments, magnetized polymer microbeads, such as Dynabeads®, are reversibly coupled to a specific cell type by a monoclonal cell-surface receptor antibody. In some embodiments, amniotic epithelial cells comprising cell surface receptors CD326, are separated and removed from cellular component 104 utilizing magnetized polymer microbeads coupled to monoclonal antibodies to the CD326.

In some embodiments, first cell type 120 comprises an epithelial stem cell. In some embodiments, first cell type 120 comprises a mesenchymal stem cell. In some embodiments, cellular component 104 is substantially depleted of mesenchymal cells. In some embodiments, cellular component 104 is substantially depleted of epithelial cells.

Following separation from cellular component 104, a first cell type is diluted with a suitable buffer solution. In some embodiments, a cell count per unit volume of a suspension in the buffer solution is determined using techniques known in the art. The suspension of first cell type 120 is further diluted to a desired cell count per unit volume by adding a volume of additional buffer solution necessary to achieve the desired cell count. In some embodiments, second cell type 121 is diluted to a desired cell count per unit volume using a suitable buffer solution.

In some embodiments, first cell type 120 is “primed” for accelerated differentiation into a differentiated cell within the recipient tissue, such as a chondrocyte wherein the recipient tissue is, for example, a knee-joint meniscus or a motor neuron wherein the recipient tissue is the spinal cord, by subjecting first cell subtype 120 to relative hypoxia. For example, in some embodiments, first cell subtype 120 is maintained at an ambient O₂ concentration of 2% for greater than about one (1) hour and less than about twenty-four (24) hours in an open container containing any appropriate cell culture media known to those in the art and placed in a 37° humidified incubator with less than about 5% CO₂ concentration. This protocol is by example only, alternative protocols for incubating stem cells under low oxygen tension are known to those with skill in the art and are used, in some embodiments.

Amniotic fluid-derived preparation 100 is formed by adding first cell type 120, first protein fraction 110, and fluid 130. In some embodiments, second cell type 121 is also added. In some embodiments, second protein fraction 111 is also added. In some embodiments, exosome component 125 is also added.

Fluid 130, in some embodiments, is a buffer solution, a cryoprotectant, another non-cytotoxic fluid, or any combination thereof. In some embodiments, fluid 130 comprises a buffered isotonic solution. A non-limiting example of a buffered isotonic solution is “Plasma-Lyte A,” manufactured by Baxter International, Inc., Deerfield, Ill. In some embodiments, fluid 130 comprises a cryopreservative, such as CryoStor CS-10, a 10% solution of dimethylsulfoxide (“DMSO”) manufactured by BioLife Solutions, Inc., Bothel, Wash. In some embodiments, fluid 130 comprises a 5% solution of DMSO. These examples are not meant to be limiting, other examples of non-cytotoxic buffering and cryoprotectant fluids may be used, at similar or different concentrations.

Following combination of first protein fraction 110, first cell type 120, and fluid 130, final concentrations of viable cells and protein activity per unit volume of amniotic fluid-derived preparation are calculated, in some embodiments. In some embodiments, the final cell concentration is about 10,000 cells per milliliter, about 50,000 cells per milliliter, about 100,000 cells per milliliter, about 250,000 cells per milliliter, about 500,000 cells per milliliter, about 1×10⁶ cells per milliliter, about 2×10⁶ cells per milliliter, about 5×10⁶ cells per milliliter, about 7.5×10⁶ cells per milliliter, about 1×10⁷ cells per milliliter, between about 10,000 cells per milliliter and about 100,000 cells per milliliter, between about 100,000 cells per milliliter and about 1×10⁶ cells per milliliter, between about 1×10⁶ cells per milliliter and about 2×10⁶ cells per milliliter, between about 2×10⁶ cells per milliliter and about 5×10⁶ cells per milliliter, or between about 5×10⁶ cells per milliliter and about 1×10⁷ cells per milliliter. In some embodiments, the final cell concentration is between zero (0) and 1.5 million cells per milliliter. In some embodiments, the final cell concentration is greater than 2.5 million cells per milliliter. In some embodiments, the final cell concentration is between 1.5 and 2.5 million cells per milliliter. In some embodiments, the final cell concentration is greater than two (2) million cells per milliliter.

In some embodiments, a small quantity of amniotic fluid-derived preparation 100 is drawn into a sterile 2 cc syringe and extruded through a 25 gauge needle to ensure amniotic fluid-derived preparation 100 is sufficiently fluid to be percutaneously or intraoperatively injected into a recipient tissue bed. In some embodiments, the final biologic activity is adjusted by adding additional fluid 130 to an end-user's pre-ordered requirements based upon the intended use of amniotic fluid-derived preparation 100. In some embodiments, the final cell concentration is adjusted by adding additional fluid 130 to an end-user's pre-ordered requirements based upon the intended use of amniotic fluid-derived preparation 100.

It is useful to employ amniotic fluid-derived preparations of different viscosities for clinical use with knowledge of expected results based upon reproducibility. Variations in viscosity affect the tendency of the amniotic fluid-derived preparation to remain and engraft a fraction of the cellular component at the site of placement. Differences in viscosity are considered based upon the intended use of the amniotic fluid-derived preparation. Some embodiments of amniotic fluid-derived preparation 100 are formed in three reproducible, standardized viscosities: high viscosity; medium viscosity; and low viscosity. Consequently, in some embodiments, the viscosity of amniotic fluid-derived preparation 100 is adjusted by mixing an additional measured quantity of fluid 130 with amniotic fluid-derived preparation 100 and calculating the final adjusted biologic activity and cell count per ml accordingly.

In some embodiments wherein high viscosity amniotic fluid-derived preparation 100 is desired, a measured quantity of biologic “thickening agent” is added to increase the viscosity of formed amniotic fluid-derived preparation 100. In some embodiments, an aqueous “hydrogel” is added to amniotic fluid-derived preparation 100, such as alginate, hyaluronic acid, gelatin, and the like, in some embodiments.

High-viscosity amniotic fluid-derived preparation 100 has a measured viscosity of greater than 10,000 centipoise (“cP”) and is formed by adding a biologically compatible thickening agent to amniotic fluid-derived preparation 100. High-viscosity amniotic fluid-derived preparation 100, in some embodiments, is a solid gel. In some embodiments, high-viscosity amniotic fluid-derived preparation 100 is a very thick fluid which is a fluid thicker than about the thickness of honey (for example, about 2000 cP to about 10,000 cP). Some examples of applications where high-viscosity amniotic fluid-derived preparation 100 may be used include the non-invasive or minimally-invasive treatment of entero-cutaneous, entero-vaginal, entero-enteric, broncho-pleural, tracheal-esophageal fistulas; graft-repair of osteochondral defects in the knee, hop, ankle, wrist, hand, and other joints; microfractures and small facial fractures; and seeding of a biocompatible extracellular scaffold for filling of large bone tissue voids following trauma, ischemic or radiation necrosis, congenital abnormalities, and surgical treatment of certain cancers.

Medium-viscosity amniotic fluid-derived preparation 100 has a measured viscosity of between 100 cP and 10,000 cP and is formed by adding a biologically compatible thickening agent to amniotic fluid-derived preparation 100. In some embodiments, medium-viscosity amniotic fluid-derived preparation 100 is a fluid with a thickness between about the thickness of motor oil and about the thickness of honey. Examples of applications where medium-viscosity amniotic fluid-derived preparation 100 may be used include treatment of wound sinus tracts, grafting of cutaneous and soft-tissue defects resulting from deep thermal or radiation burns; spinal and other bony fusion procedures (when combined with currently available bone putty or as a stand-alone application into a cervical or lumbar intervertebral spacer); facial trauma and facial fracture treatment; bone grafting; alveolar cleft (“cleft palate”) grafting; treatment of dental/tooth tissue defects; chronic inflammatory bursitis; intervertebral facet-based pain; tears of the meniscal cartilage; application to entero-entero and other surgical anastomoses; treatment of non-union and mal-union of fractures, intra-peritoneal application following surgical adhesiolysis; intra-peritenon implantation following Achilles' tendon debridement and anastamotic repair; defects of the calvarium following trauma; emergency decompressive craniotomy; surgical breast reconstruction; and following acetabular and other articular joint surface resurfacing, for example.

Low-viscosity amniotic fluid-derived preparation 100 has a measured viscosity of less than 100 cP (for example a viscosity between about 0 cP and about 100 cP) and is formed, in some embodiments, by adding a biologically compatible thickening agent to amniotic fluid-derived preparation 100. In some embodiments, low-viscosity amniotic fluid-derived preparation 100 is formed by adding additional fluid 130 to amniotic fluid-derived preparation 100. Low-viscosity amniotic fluid-derived preparation 100 may be easily injected through a hypodermic needle larger than 25G and is, therefore, useful in clinical applications wherein preparation 100 is delivered to the target tissue site by injection. Examples of applications where low-viscosity amniotic fluid-derived preparation may be used include treatment of chronic wounds, radiation burns, and thermal injury by subcutaneous injection; injection into peri-rotator cuff soft tissues following rotator cuff repair; injection to facilitate non-surgical repair and healing of supraspinatus, infraspinatus, teres minor, and subscapularis tears; other muscle, ligament, tendon, and soft-tissue tears; epicondylitis; and other similarly debilitating chronic fascial inflammatory conditions such as plantar fasciitis or fasciolosis.

In some embodiments, amniotic fluid-derived preparation 100 is packaged with standardized ranges of any one quantity or combination of quantities of first protein fraction 110 activity, second protein fraction 111 activity, first cell type 120 concentration, viable first cell type 120 concentration, second cell type 121 concentration, viable second cell type 121 concentration, and degree of viscosity based upon the mode used for delivery (injection versus intraoperative application, recipient host tissue type, other specific requirements, for example) and intended therapeutic use.

The completed amniotic fluid-derived preparation is sealed in packaging vials and frozen for storage at minus eighty (−80) degrees Celsius, in some embodiments.

### INCORPORATION BY REFERENCE

The entire disclosure of each of the patent documents and scientific articles cited herein is incorporated by reference for all purposes.

### EQUIVALENTS

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above, and are intended to fall within the scope of the appended claims. The disclosure can be embodied in other specific forms without departing from the essential characteristics thereof. The scope of the disclosure is indicated by the appended claims rather than by the foregoing description, and all changes that come within the meaning and range of equivalency of the claims are intended to be embraced therein.

What is claimed is:

1. An amniotic fluid-derived preparation comprising:
a first acellular or cell-depleted protein fraction isolated from a donor amniotic fluid;
a cellular component isolated from the donor amniotic fluid; and
a fluid, wherein the fluid dilutes the first protein fraction and the cellular component.

2. The amniotic fluid-derived preparation of claim 1, wherein the donor amniotic fluid is a human amniotic fluid.

3. The amniotic fluid-derived preparation of claim 1, wherein the donor amniotic fluid comprises a non-human donor amniotic fluid.

4. The amniotic fluid-derived preparation of claim 1, further comprising a cryopreservative.

5. The amniotic fluid-derived preparation of claim 4, wherein the cryopreservative comprises dimethylsulfoxide.

6. The amniotic fluid-derived preparation of claim 4, wherein the cryopreservative comprises glycerol.

7. The amniotic fluid-derived preparation of claim 1, wherein the first acellular or cell-depleted protein fraction comprises an amniotic fluid proteome.

8. The amniotic fluid-derived preparation of claim 7, wherein the first acellular or cell-depleted protein fraction comprises a secondary source protein, and
wherein the amniotic fluid proteome does not comprise the secondary source protein.

9. The amniotic fluid-derived preparation of claim 1, wherein the first acellular or cell-depleted protein fraction comprises a regulatory protein taken from the group of regulatory proteins consisting of a growth factor, a signaling ligand, a receptor molecule, a cytokine, a transcriptional regulator, and an immune regulator.

10. The amniotic fluid-derived preparation of claim 1, wherein the first acellular or cell-depleted protein fraction comprises a concentrated enzyme.

11. The amniotic fluid-derived preparation of claim 1, wherein the first acellular or cell-depleted protein fraction comprises a concentrated binding protein.

12. The amniotic fluid-derived preparation of claim 1, wherein the first acellular or cell-depleted protein fraction comprises a concentrated carrier protein.

13. The amniotic fluid-derived preparation of claim 1, wherein the cellular component comprises an epithelial stem cell.

14. The amniotic fluid-derived preparation of claim 1, wherein the cellular component comprises a mesenchymal stem cell.

15. The amniotic fluid-derived preparation of claim 1, wherein the cellular component comprises a progenitor cell.

16. The amniotic fluid-derived preparation of claim 1, wherein the cellular component comprises an epithelial cell.

17. The amniotic fluid-derived preparation of claim 1, wherein the cellular component is substantially depleted of epithelial cells.

18. The amniotic fluid-derived preparation of claim 1, wherein the cellular component is substantially depleted of mesenchymal cells.

19. The amniotic fluid-derived preparation of claim 1, wherein the first acellular or cell-depleted protein fraction isolated from a donor amniotic fluid is acellular.

20. An amniotic fluid derivative comprising:
a concentrated cellular component;
an acellular or cell-depleted supernatant; and
a fluid, wherein the fluid dilutes the concentrated cellular component and the supernatant.

21. The amniotic fluid derivative of claim 20, further comprising a concentrated exosome component.

22. The amniotic fluid derivative of claim 20, wherein the supernatant is substantially free of exosomes.

23. The amniotic fluid derivative of claim 20, wherein the concentrated cellular component comprises a non-amniotic fluid derived cell.

24. The amniotic fluid derivative of claim 20, wherein the supernatant is substantially depleted of albumin.

25. The amniotic fluid derivative of claim 20, wherein the supernatant is substantially depleted of one or more immunoglobulins.

26. The amniotic fluid derivative of claim 20, wherein the supernatant is acellular.

27. A set of amniotic fluid-derived preparations, wherein each amniotic fluid-derived preparation comprises:
a first acellular or cell-depleted protein fraction isolated from a donor amniotic fluid;
a cellular component isolated from the donor amniotic fluid; and
a fluid, wherein the fluid dilutes the first acellular or cell-depleted protein fraction and the cellular component, and the dilution of the first acellular or cell-depleted protein fraction and the cellular component in each amniotic fluid-derived preparation in the set is the same or about the same as the dilution of the first acellular or cell-depleted protein fraction and the cellular component in every other amniotic fluid-derived preparation in the set.

28. The set of claim 27, comprising at least 10 amniotic fluid-derived preparations.

29. The set of claim 27, comprising at least 50 amniotic fluid-derived preparations.

30. A set of amniotic fluid-derived preparations, wherein each amniotic fluid-derived preparation comprises:
a concentrated cellular component;
an acellular or cell-depleted supernatant; and
a fluid, wherein the fluid dilutes the concentrated cellular component and the acellular or cell-depleted supernatant, and the dilution of the concentrated cellular component and the supernatant in each amniotic fluid-derived preparation in the set is the same or about the same as the dilution of the concentrated cellular component and the acellular or cell-depleted supernatant in every other amniotic fluid-derived preparation in the set.

31. The set of claim 30, comprising at least 10 amniotic fluid-derived preparations.

32. The set of claim 30, comprising at least 50 amniotic fluid-derived preparations.


SEQUENCE LISTING (1 sequence)

SEQ ID NO: 1 - protein, 609 aa, Homo sapiens [sequence omitted]